(19) United States
(12) Patent Application Publication
(10) Pub. No.: US 2010/0111886 A1
(43) Pub. Date: May 6, 2010

(54) HAIR STYLING COMPOSITION

(75) Inventors: Gilles Verboom, St. Charles, IL (US); Michael F. Konieczny, Palatine, IL (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: ALBERTO-CULVER COMPANY, Melrose Park, IL (US)

(21) Appl. No.: 12/519,111

(22) PCT Filed: Dec. 13, 2007
(86) PCT No.: PCT/US07/87356

§ 371 Date: Dec. 11, 2009

Related U.S. Application Data
(60) Provisional application No. 60/869,856, filed on Dec. 13, 2006.

(51) Int. Cl. A61K 8/81; A61Q 5/06; A61K 8/73

(52) U.S. Cl. 424/70.122; 424/70.15; 424/70.13

(57) ABSTRACT

Provided is a hair-styling composition, which preferably includes polyvinylpyrrolidone, at least one additional film former, at least one thickener and water, wherein the composition is substantially free of carbomer, and when applied to human hair, exhibits at least about 80% curl retention after about 2 hours under conditions of about 85% relative humidity and about 75° F. Also provided is a method of styling keratinous fibers with the composition of the present invention and a method of controlling the frizz condition of keratinous fibers with the composition.

### BACKGROUND OF THE INVENTION

Hair styling or hair setting compositions are widely used by consumers in the cosmetic industry to retain a particular shape or style of the hair. Hair styling compositions can assist in manipulating (styling) the hair, providing temporary benefits in holding the shape of the hairstyle (fixing) and/or maintaining the shine or appearance (grooming, restyling) of the hair, e.g., in the evening, during the day, between hair washing periods, or between subsequent hair setting procedures.

Various objective and subjective methods are used to measure the efficacy of a hair-styling composition. One method commonly employed to test the efficacy of hair styling compositions involves measuring curl retention under conditions of high humidity. In this methodology, hair tresses are curled either before or after applying the hair styling product to the hair, and the ability of the composition to retain curl (curl retention) can be monitored periodically during exposure to a controlled temperature and relative humidity (RH). For instance, the retention of hair rolled into curls of ¾ inch diameter prepared by applying 0.3 ml of styling gel onto 1.8 gm hair tresses can be measured under controlled temperatures, e.g., in the range of about 73° F. to about 81° F., and high relative humidity, e.g., in the range of about 80-90% RH. It is sometimes referred to as high humidity curl retention (HHCR).

Hair styling compositions also can be subjectively evaluated, e.g., by visual and tactile sensory methods (e.g., by visual examination and touching) for characteristics such as appearance (shine, cleanliness, naturalness of appearance and texture), feel (stiffness, tackiness, softness), curl memory (bounce, and restylability), ease of combing and brushing the hair, residue (flaking), static, smoothness, and the like. Also of importance are the aesthetic characteristics and appearance provided by hair styling compositions before, during, and after application to hair. Preferably, the product viscosity should be non-runny to avoid dripping during application. The product should be easy to spread, have a smooth texture, a non-tacky feel, and be able to dry relatively quickly on the hair.

Of further importance is the ability of hair styling compositions to control hair “frizz.” Hair is often subjected to a wide variety of stresses that can cause damage to the hair. These include shampooing, rinsing, drying, heating, combing, styling, perming, coloring, exposure to the elements, and the like. Such practices can leave the hair in a dry, rough, lusterless, or frizzy condition, which can be caused, e.g., by repeated abrasion of the hair surface and removal of the hair's natural oils and other natural conditioning and moisturizing components. Additionally, hair is often subjected to weather-related stresses, e.g., sunlight, wind, and changes in temperature and humidity, which can cause hair fizz and other conditions considered by consumers to be cosmetically undesirable.

Most commercial hair-setting compositions include hair-setting polymers (styling or fixative), viscosity-increasing polymers, and polymer modifiers, in addition to solvents, co-solvents, and cosmetic adjuvants, such as preservatives, color, fragrance, and the like. Polyvinylpyrrolidone (PVP), a film former, which has been widely used as a fixative in hair styling compositions, imparts particularly desirable subjective properties to the hair. However, conventional hair setting formulations that utilize PVP provide only marginal humidity resistance, typically only providing an HHCR of about 70% of the initial curl for a period of not more than about 0.75 hours. In order to provide hair styling compositions with improved HHCR, the conventional approach has been to replace PVP with certain moisture resistant hair setting polymers. While such polymers can increase the HHCR, they tend to impart an unnatural stiffness to the hair, and fail to provide the desirable subjective properties that PVP provides, such as smoothness and shine.

In view of the drawbacks associated with conventional hair styling compositions, there is an ongoing need for hair styling compositions that provide the benefits of PVP, and yet provide excellent HHCR and resistance to frizziness. The present invention provides such compositions and methods of using such compositions

### BRIEF SUMMARY OF THE INVENTION

The present invention provides a hair-styling composition that includes polyvinylpyrrolidone, at least one additional film former, at least one thickener and water, wherein the composition is substantially free of carbomer and, when applied to human hair, exhibits at least about 80% curl retention after about 2 hours under conditions of about 85% relative humidity and about 75° F. The present invention also provides a method of styling keratinous fibers, which method includes applying the composition of the present invention in an amount effective for styling. The present invention further provides a method of controlling frizz in keratinous fibers, which method includes applying the composition of the present invention in an amount effective to control fizz.

### DETAILED DESCRIPTION OF THE INVENTION

The present invention provides a hair-styling composition that includes polyvinylpyrrolidone, at least one additional film former, at least one thickener and water, wherein the composition is substantially free of carbomer and, when applied to human hair, exhibits at least about 80% curl retention after about 2 hours under conditions of about 85% relative humidity and about 75° F. The hair-styling composition of the present invention can further include one or more additional ingredients such as, for example, a conditioning agent, a film modifier, an anionic copolymer, and the like, and combinations thereof.

The composition of the present invention includes polyvinylpyrrolidone in an amount effective to provide the hair with desirable subjective properties such as, e.g., glossy, rigid, low flaking, smooth, and yet provides the hair with an uncharacteristically high HHCR as compared with conventional PVP hair styling compositions. Preferably, the composition of the present invention includes polyvinylpyrrolidone in an amount of from about 0.01 wt % to about 20 wt %, e.g., from about 0.05 wt % to about 15 wt % polyvinylpyrrolidone, e.g., from about 0.1 wt % to about 10 wt % polyvinylpyrrolidone.

The composition of the present invention also includes one or more additional film formers, which can include combinations of one or more film formers (in addition to PVP). Suitable additional film formers can include, e.g., vinylpyrrolidone copolymers, cationic cellulose derivatives, polyurethanes, acrylates/hydroxyester acrylate copolymer, celluloses and polysaccharide gums and their derivatives and the like, and combinations thereof. The composition of the present invention can include any suitable amount of additional film former, for example, from about 0.01 wt % to about 20 wt % of one or more additional film formers, from about 0.05 wt % to about 15 wt % of one or more additional film formers, or from about 0.1 wt % to about 10 wt % of one or more additional film formers.

Exemplary film forming vinylpyrrolidone copolymers include one or more nonionic copolymers comprising a combination of N-vinylpyrrolidone, methacrylamide and N-vinylimidazole monomeric units. Preferably, the composition of the present invention includes from about 0.01 wt % to about 20 wt % of one or more film forming vinylpyrrolidone copolymers, e.g., from about 0.05 wt % to about 15 wt % of one or more film forming vinylpyrrolidone copolymers, or from about 0.1 wt % to about 10 wt % of one or more film forming vinylpyrrolidone copolymers. An exemplary film forming vinylpyrrolidone copolymer is LUVISET® CLEAR, available from BASF.

The additional film former also can include a cationic cellulose derivative. Any suitable amount of a cationic cellulose derivative can be included in the composition of the present invention, for example, from about 0.01 wt % to about 20 wt % of one or more cationic cellulose derivatives, from about 0.02 wt % to about 15 wt % of one or more cationic cellulose derivatives, or from about 0.05 wt % to about 10 wt % of one or more cationic cellulose derivatives. A preferred class of cationic cellulose derivatives includes cellulosic polymers comprising cationic substituents derived from the polymerization or oligomerization of dimethyl diallyl ammonium chloride. An exemplary cationic cellulose derivative is CELQUAT® H-100, available from National Starch and Chemical.

The hair styling composition of the present invention is substantially free of carbomer. Carbomer is defined by the Cosmetic Toiletries and Fragrance Association as a homopolymer of acrylic acid crosslinked with an alkyl ether of pentaerythritol, an allyl ether of sucrose, or an allyl ether of propylene. As used herein, “substantially free” of carbomer means that the composition of the present invention includes no more than about 0.5 wt % carbomer, more preferably no more than about 0.1 wt % carbomer, and most preferably no more than about 0.05 wt % carbomer (e.g., less than 0.05 wt % carbomer or no carbomer at all).

Any suitable thickener, excluding carbomer, can be used in the composition of the present invention. Suitable thickeners can include, e.g., one or more associative and non-associative thickeners, one or more polysaccharides, their gums and their derivatives, and the like, and combinations thereof. Exemplary associative thickeners can include an acrylates/beheneth-25 acrylate copolymer, a PEG-180/Dodoxynol-5/PEG-25 tristyrylphenol/tetramethoxymethylglycouril copolymer, and combinations thereof. Any suitable amount of thickener can be included in the composition of the present invention, for example, from about 0.01 wt % to about 15 wt % of one or more thickeners, from about 0.05 wt % to about 8.0 wt % of one or more thickeners, or from about 0.1 wt % to about 3.0 wt % of one or more thickeners. An exemplary thickener includes a combination of the following associative thickeners: TINOVIS® GTC, available from Ciba Specialty Chemicals, and PURE THIX® HH, available from Southern Clay.

The composition of the present invention further includes an aqueous carrier, e.g., water. Any suitable quantity of water can be used in the composition of the present invention, e.g., from about 60 wt % to about 97 wt % water (e.g., from about 85% to about 95% water). Preferably, the composition of the present invention includes from about 60 wt % to about 97 wt %, and more preferably from about 70 wt % to about 90 wt %, and most preferably from about 80 wt % to about 90 wt % water. Preferably, the water used in the composition of the present invention is deionized water.

The composition of the present invention can further include a conditioning agent. Suitable conditioning agents can include, for example, one or more amphoteric copolymers, one or more amphoteric terpolymers, one or more cationic conditioners and the like, and combinations thereof. Exemplary conditioning agents include amphoteric terpolymers of acrylic acid, diallyl dimethyl ammonium chloride, and acrylamide. The composition of the present invention can include any suitable amount of conditioning agent, for example, from about 0.01 wt % to about 20 wt % of one or more conditioning agents, from about 0.01 wt % to about 15 wt % of one or more conditioning agents, or from about 0.05 wt % to about 10 wt % of one or more conditioning agents. An exemplary conditioning agent is polyquaternium-39, sold under the tradename MERQUAT® PLUS 3330, available from Nalco Co.

The composition of the present invention can further include a film modifier. Suitable film modifiers can include, for example, one or more aminosilicones, one or more PEG-n dimethicones, one or more plasticizers, for example, known in the art as glycols, glycol ethers, glycerine, and the like, and combinations thereof. Any suitable aminosilicone can be used in the composition of the present invention. Exemplary aminosilicones can include one or more compounds such as: a polyoxyalkylenated aminosilicone polymer of the B(AB)ₙ type, wherein A is a polysiloxane block and B is a polyoxyalkylenated block, comprising repeating units of the formulae: [SiMe₂-O—(SiMe₂O)ₓSiMe₂-R′—N(H)—R″—O(C₂H₄O)ₐ—(C₃H₆O)_{b}—R″—N(H)—R′—], wherein: (i) a is from about 1 to about 200, (ii) b is from 0 to about 200, (iii) each R′, which may be identical or different, is a divalent organic group which is linked to the adjacent silicon atom via a carbon-silicon bond and to the nitrogen atom, (iv) each R″, which may be identical or different, is a divalent organic group which is linked to the adjacent oxygen atom via a carbon-oxygen bond and to the nitrogen atom, and (v) x is chosen such that the siloxane block represents between 50 and 95 mol % relative to the total weight of the polyoxyalkylenated aminosilicone polymer; or a combination thereof. The composition of the present invention can include any suitable amount of aminosilicone, for example, from about 0.01 wt % to about 10.0 wt % of one or more aminosilicones, from about 0.01 wt % to about 5.0 wt % of one or more aminosilicones, or from about 0.05 wt % to about 2.0 wt % of one or more aminosilicones. An exemplary aminosilicone is SILSOFT® A-843, available from GE Silicones.

Suitable film modifiers also can include one or more PEG-n dimethicones. Suitable PEG-n dimethicones include polyethylene glycol derivatives of dimethicone containing an average of n moles of ethylene oxide, e.g., where n is in the range of about 3 to about 16. The composition of the present invention can include any suitable amount of a PEG-n dimethicone, for example, from about 0.01 wt % to about 10.0 wt % of one or more PEG-n dimethicones, from about 0.01 wt % to about 5.0 wt % of one or more PEG-n dimethicones, or from about 0.05 wt % to about 2.0 wt % of one or more PEG-n dimethicones. An exemplary PEG-n dimethicone is a PEG-12 dimethicone, available from Dow Corning under the trade name DC-193.

The composition of the present invention can further include an anionic copolymer. Exemplary anionic copolymers include copolymers of an acrylamidomethyl propane sulfonic acid or a salt thereof and another anionic monomer. Preferably, the anionic copolymer comprises from about 90 mole percent to about 20 mole percent of a first anionic monomer and from about 10 mole percent to about 80 mole percent of a second anionic monomer, which is 2-acrylamido-2-methyl-1-propanesulfonic acid or a salt thereof, e.g., a base addition salt thereof. More preferably, the anionic copolymer includes monomers of methacrylic acid or a salt thereof, and 2-acrylamido-2-methyl-1-propanesulfonic acid or a salt thereof. The composition of the present invention can include any suitable amount of an anionic copolymer, for example, from about 0.01 wt % to about 20 wt % of one or more anionic copolymers, from about 0.01 wt % to about 15 wt % of one or more anionic copolymers, or from about 0.05 wt % to about 10 wt % of one or more anionic copolymers. An exemplary anionic copolymer is FIXOMER® A-30, available from Nalco Co.

The hair styling composition of the present invention can include other components used in hair styling, hair fixative, hair setting, and/or hair grooming gels, rinses, emulsions (oil-in-water, water-in-oil or multiphase), lotions, creams, pomades, sprays (pressurized or non-pressurized), spritzes, mousses, foams, shampoos, and solids (e.g., as sticks, semisolids and the like).

Preferably, the hair-styling composition of the present invention exhibits at least about 80% curl retention after about 4 hours under conditions of about 85% relative humidity and about 75° F. More preferably, the hair-styling composition of the present invention exhibits at least about 80% curl retention after about 7 hours under conditions of about 85% relative humidity and about 75° F. Most preferably, the hair-styling composition of the present invention exhibits at least about 60% curl retention after about 24 hours under conditions of about 85% relative humidity and about 75° F.

The present invention further provides a method of styling keratinous fibers, which preferably includes applying to the keratinous fibers a styling-effective amount of the composition of the present invention. The keratinous fibers can include mammalian hair, e.g., human hair. The composition can be applied in any suitable manner, e.g., by working the composition through the hair, e.g., with the hands and fingers or with a suitable implement such as, e.g., a comb or brush, to ensure uniform coverage.

The present invention further provides a method of controlling frizz, which preferably includes applying to the keratinous fibers a frizz controlling effective amount of the composition of the present invention. Consumers often associate frizz, e.g., the tendency of individual keratinous fibers (e.g., individual hairs) to stray from alignment or conformity with one another, with an unruly or undesirable appearance. The styling composition of the present invention can be use to effectively control (e.g., inhibit, reduce or ameliorate) frizz in keratinous fibers, e.g., human hair.

The following example further illustrates the invention but, of course, should not be construed as in any way limiting its scope.

### Example 1

This example illustrates a process for preparing an exemplary composition of the present invention.

The list of ingredients and the amount of each ingredient (in wt % relative to the total weight of the composition) of hair styling composition are summarized in Table 1. The preparation of Hair Styling Composition consists of the following process. Phases A, B, and C are separately prepared. When phase C becomes clear, it is added to phase B. Finally, phase A is added to phases B and C with mixing.

TABLE 1

| Hair Styling Composition | Phase | (wt. %) |
|---|---|---|
| Deionized water | A | 46.49 |
| Cationic cellulose derivative | A | 0.50 |
| Acrylates/beheneth-25 acrylate copolymer | A | 2.00 |
| Deionized water | B | q.s. to 100 |
| PVP (20% solution) | B | 4.00 |
| PEG-180/Dodoxynol-5/PEG-25 | B | 0.20 |
| tristyrylphenol/tetramethoxymethylglycouril | | |
| copolymer | | |
| DMDM hydantoin | B | 0.50 |
| Tetrasodium EDTA | B | 0.05 |
| Aminomethylpropanol (95%) | B | 0.50 |
| Vinylpyrrolidone copolymer (20%) | B | 3.50 |
| Vitamin Blend | B | 0.0001 |
| Propylene glycol | C | 1.00 |
| Sorbitol (70%) | C | 0.50 |
| Film modifier | C | 0.10 |
| Polyquaternium-39 | C | 0.50 |
| Fragrance | C | 0.075 |
| PEG-40 hydrogenated castor oil | C | 0.20 |

### Example 2

Using the method described in Example 1, the Hair Styling Composition described in Table 2 was prepared.

TABLE 2

| Hair Styling Composition | Phase | (wt. %) |
|---|---|---|
| Deionized water | A | 45.00 |
| Cationic cellulose derivative | A | 0.50 |
| Acrylates/beheneth-25 acrylate copolymer | A | 2.00 |
| Deionized water | B | q.s. to 100 |
| PVP (20% solution) | B | 4.00 |
| PEG-180/Dodoxynol-5/PEG-25 | B | 0.20 |
| tristyrylphenol/tetramethoxymethylglycouril | | |
| copolymer | | |
| DMDM hydantoin | B | 0.50 |
| Tetrasodium EDTA | B | 0.05 |
| Aminomethylpropanol (95%) | B | 0.40 |
| Vinylpyrrolidone copolymer (20%) | B | 3.50 |
| Anionic copolymer | B | 1.00 |
| Vitamin Blend | B | 0.0001 |
| Propylene glycol | C | 1.00 |
| Sorbitol (70%) | C | 0.50 |
| Film modifier | C | 0.10 |
| Polyquaternium-39 | C | 0.50 |
| Fragrance | C | 0.075 |
| PEG-40 hydrogenated castor oil | C | 0.20 |

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context

1. A hair-styling composition comprising polyvinylpyrrolidone, at least one additional film former, at least one thickener, and water, wherein the composition is substantially free of carbomer and, when applied to human hair, exhibits at least about 80% curl retention after about 2 hours under conditions of about 85% relative humidity and about 75° F.

2. The hair-styling composition of claim 1, wherein the polyvinylpyrrolidone is present in the composition in an amount from about 0.01 wt % to about 20 wt %.

3. The hair-styling composition of claim 1, wherein the additional film former is a vinylpyrrolidone copolymer.

4. The hair-styling composition of claim 3, wherein the vinylpyrrolidone copolymer is a nonionic polymer comprising N-vinylpyrrolidone, methacrylamide and N-vinylimidazole monomeric units.

5. The hair-styling composition of claim 4, wherein the vinylpyrrolidone copolymer is present in the composition in an amount from about 0.01 wt % to about 20 wt %.

6. The hair-styling composition of claim 1, wherein the additional film former is a cationic cellulose derivative.

7. The hair-styling composition of claim 6, wherein the cationic cellulose derivative is a cellulosic polymer comprising cationic substituents derived from the polymerization or oligomerization of dimethyl diallyl ammonium chloride.

8. The hair-styling composition of claim 7, wherein the cationic cellulose derivative is present in the composition in an amount from about 0.01 wt % to about 20 wt %.

9. The hair-styling composition of claim 1, wherein the thickener comprises one or more associative thickeners.

10. The hair-styling composition of claim 9, wherein the thickener includes an acrylates/beheneth-25 acrylate copolymer, a PEG-180/Dodoxynol-5/PEG-25 tristyrylphenol/tetramethoxymethylglycouril copolymer, or a combination thereof.

11. The hair-styling composition of claim 10, wherein the thickener is a mixture of an acrylates/beheneth-25 acrylate copolymer and PEG-180/Dodoxynol-5/PEG-25 tristyrylphenol/tetramethoxymethylglycouril copolymer, and the mixture is present in the composition in an amount from about 0.01 wt % to about 15 wt %.

12. The hair-styling composition of claim 1, further comprising a conditioning agent.

13. The hair-styling composition of claim 12, wherein the conditioning agent comprises an amphoteric terpolymer.

14. The hair-styling composition of claim 13, wherein the amphoteric terpolymer is a terpolymer of acrylic acid, diallyl dimethyl ammonium chloride and acrylamide.

15. The hair-styling composition of claim 13, wherein the amphoteric terpolymer is present in the composition in an amount from about 0.01 wt % to about 20 wt %.

16. The hair-styling composition of claim 1, further comprising a film modifier.

17. The hair styling composition of claim 16, wherein the film modifier comprises one or more aminosilicones.

18. The hair-styling composition of claim 17, wherein the aminosilicone is a polyoxyalkylenated aminosilicone polymer of the B(AB)ₙ type, wherein A is a polysiloxane block and B is a polyoxyalkylenated block, comprising repeating units of the formulae:
[SiMe₂-O—(SiMe₂O)_{x′″}SiMe₂-R′—N(H)—R″—O(C₂H₄O)ₐ—(C₃H₆O)_{b}—R″—N(H)—R′—], wherein:
a is from about 1 to about 200,
b is from 0 to about 200,
R′, which may be identical or different, is a divalent organic group which is linked to the adjacent silicon atom via a carbon-silicon bond and to the nitrogen atom,
R″, which may be identical or different, is a divalent organic group which is linked to the adjacent oxygen atom via a carbon-oxygen bond and to the nitrogen atom, and
x′″ is chosen such that the siloxane block represents between 50 and 95 mol % relative to the total weight of the polyoxyalkylenated aminosilicone polymer; or a combination thereof.

19. The hair-styling composition of claim 16, wherein the film modifier is a PEG-n dimethicone.

20. The hair styling composition of claim 19, wherein the PEG-n dimethicone is PEG-12 dimethicone.

21. The hair-styling composition of claim 1, further comprising an anionic copolymer.

22. The hair-styling composition of claim 21, wherein the anionic copolymer comprises monomers of an acrylamidomethyl propane sulfonic acid or a salt thereof.

23. The hair-styling composition of claim 22, wherein the anionic copolymer comprises from about 10 mole percent to about 80 mole percent monomers of 2-acrylamido-2-methyl-1-propanesulfonic acid or a salt thereof.

24. The hair-styling composition of claim 23, wherein the anionic copolymer is present in the composition in an amount from about 0.01 wt % to about 20 wt %.

25. The hair-styling composition of claim 24, wherein the anionic polymer includes monomers of methacrylic acid or a salt thereof and monomers of 2-acrylamido-2-methyl-1-propanesulfonic acid or a salt thereof.

26. The hair-styling composition of claim 1, comprising from about 60 wt % to about 97 wt % water.

27. A hair-styling composition, in the form of a gel, comprising polyvinylpyrrolidone, a vinylpyrrolidone copolymer, a thickener, a cationic cellulose derivative, a conditioning agent, an amino silicone and water, wherein the composition is substantially free of carbomer and, when applied to human hair, exhibits at least about 80% curl retention after about 2 hours under conditions of about 85% relative humidity and about 75° F.

28. A hair-styling composition, in the form of a gel, comprising polyvinylpyrrolidone, a vinylpyrrolidone copolymer, a thickener, a cationic cellulose derivative, a conditioning agent, PEG-12 dimethicone and water, wherein the composition is substantially free of carbomer and, when applied to human hair, exhibits at least about 80% curl retention after about 2 hours under conditions of about 85% relative humidity and about 75° F.

29. The hair-styling composition of claim 1, wherein the composition exhibits at least about 80% curl retention after about 4 hours under conditions of about 85% relative humidity and about 75° F.

30. The hair-styling composition of claim 1, wherein the composition exhibits at least about 80% curl retention after about 7 hours under conditions of about 85% relative humidity and about 75° F.

31. (canceled)

32. A method of styling keratinous fibers, the method comprising contacting the keratinous fibers with a styling-effective amount of the composition of claim 1 and styling keratinous fibers.

33. The method of claim 32, wherein the keratinous fibers comprise mammalian hair.

34. The method of claim 33, wherein the keratinous fibers comprise human hair.

35. A method of controlling frizz in keratinous fibers, the method comprising contacting the keratinous fibers with a frizz controlling effective amount of the composition of claim 1.

36. The method of claim 35, wherein the keratinous fibers comprise mammalian hair.

37. The method of claim 36, wherein the keratinous fibers comprise human hair.
